(19) United States
(12) Patent Application Publication
(10) Pub. No.: US 2007/0138088 A1
(43) Pub. Date: Jun. 21, 2007

(54) Method and composition for removing radionuclide with microorganisms

(75) Inventors: Nobuyoshi Ishii, Chiba (JP); Shigeo Uchida, Chiba (JP)

Correspondence Address: WELSH & KATZ, LTD, 120 S RIVERSIDE PLAZA, 22ND FLOOR, CHICAGO, IL, 60606, US

(73) Assignee: National Institute of Radiological Sciences, Chiba (JP)

(21) Appl. No.: 11/584,022

(22) Filed: Oct. 20, 2006

(30) Foreign Application Priority Data
Apr. 28, 2005 (JP) PCT/JP05/08149
Apr. 28, 2004 (JP) JP NO.2004-133367

(51) Int. Cl. G21F 9/20; C12N 1/12

(52) U.S. Cl. 210/601; 588/20; 435/257.4

(57) ABSTRACT

Radionuclide can be easily removed from a solution under aerobic condition by culturing Euglena in contact with radionuclide in a solution that is suitable for viability Euglena to thus transfer said radionuclide to Euglena and separating said Euglena comprising radionuclide from said solution.

### TECHNICAL FIELD

The present invention relates to a treatment of radionuclide and specifically to a treatment of radionuclide utilizing Euglena.

### BACKGROUND OF INVENTION

The effects of radionuclide on the human body and ecosystems are well known and a safe method for treating radioactive wastes is desired.

Among the various radionuclides, technetium (⁹⁹Tc), which is one isotope of technetium and has mass number of 99, is a fission product of uranium and is characterized in having an enormously long half-life, 210,000 years. Therefore, there is fear that it accumulates in natural environments and that it affects human body through the food chain. Accordingly, in the longitudinal safety evaluation of reprocessing plant or processing of radioactive waste disposal, ⁹⁹Tc is one of the most noteworthy radionuclide. ⁹⁹Tc mainly exists as pertechnetate ion (⁹⁹TcO₄⁻) in the radioactive waste or in biosphere. This chemical structure easily dissolves in water and thus it is known to easily migrate from place to place in the environment.

As a method for biologically removing this element from a solution, a method of utilizing bacterium has been studied (For example, see J. Henrot, Health Physics, Vol. 57, No. 2 (August), pp. 239-245 (1989)). A bacterium has a very small cell size and thus its specific surface area (surface area per unit weight) is very large. In other words, the area of cell surface that contacts with a solution comprising technetium is so wide and thus it is possible to efficiently remove technetium from the solution. However, the ability of bacterium to remove technetium from a solution is exerted only under anaerobic condition. Therefore, it is necessary to maintain the solution under anaerobic condition to remove technetium by bacterium. But, the cost of operation and maintenance of a device for anaerobic culture in order to maintain the solution comprising technetium under anaerobic condition would be very high. Therefore, the inventor of the present invention has studied a biological removal of technetium from a solution under aerobic condition.

### DISCLOSURE OF THE INVENTION

The purpose of the present invention is to provide a method for easily removing radionuclide from a solution under aerobic condition and a removal composition that can be utilized in said method.

The inventor of the present invention found that radionuclide can be removed by treating a solution containing the radionuclide with Euglena under aerobic condition.

The present invention provides a method for removing radionuclide from a solution characterized in use of Euglena.

The present invention also provides a method for removing radionuclide from a solution comprising: contacting Euglena with radionuclide in a solution in which Euglena is viable to thus transfer said radionuclide to Euglena and separating said Euglena containing radionuclide from said solution.

The present invention also provides a composition for removing radionuclide, comprising living cells of Euglena.

In accordance with the method and the composition of the present invention, radionuclide, in particular, technetium can be easily removed and isolated from a solution. Especially, the removal can be conducted under aerobic condition. It is not necessary to conduct shaking culture or to do maintenance of culture and thus radionuclide can be easily removed.

### BRIEF DESCRIPTION OF THE DRAWINGS

FIG. 1 shows procedures in Example.

FIG. 2 shows the results of Example.

### BEST MODE FOR CARRYING OUT THE INVENTION

The method and the composition of the present invention will be explained below.

The method of the present invention to remove radionuclide from a solution is characterized in use of Euglena. More specifically, the method of the present invention is to remove radionuclide from a solution comprising: contacting Euglena with radionuclide in a solution in which Euglena is viable to thus transfer said radionuclide to Euglena and separating said Euglena containing radionuclide from said solution.

Euglena is a genus disclosed in both the classification tables of zoology and phytology and is widely distributed in fresh water. In general, Euglena has spindle-shaped form but sometimes shows a variety of forms due to phenomenon of so-called “Euglenoid movement”. This single cell microorganism can conduct photosynthesis but also can proliferate heterotrophically. See “Euglena physiology and biochemistry”, p. 1-3, edited by Kitaoka Seisaburo, Gakkai-Syuppan-Center, Dec. 10, 1989.

In the present specification, “Euglena” includes all the species, varieties and sub species of the genus Euglena in the taxonomic classification of zoology and phytology.

Dozens of species belonging to genus Euglena are known. Examples of species that can be used in the present invention include the following specifies: Euglena acus, Euglena caudata, Euglena chadefaudii, Euglena deses, Euglena ehrenbergii, Euglena geniculata, Euglena glacilis, Euglena granulata, Euglena intermedia, Euglena mutabilis, Euglena oxyuris, Euglena pisciformis, Euglena proxima, Euglena sanguinea, Euglena sociabis, Euglena spirogyra, Euglena stellata, Euglena tripteris, Euglena viridis.

Euglena has commonly observed feature that there are many helical strips from front-end to back-end over the whole cell. In addition, the cell membrane covering outside the cell is very unique as compared with other organisms and is called as “pellicle”. Pellicle consists of plasma membrane, membrane skeleton, microtubule, fiber and the like. Endoplasmic reticula and muciferous body are distributed under the membrane skeleton. See “Euglena physiology and biochemistry”, p. 1-3, edited by Kitaoka Seisaburo, Gakkai-Syuppan-Center, Dec. 10, 1989. As described above, the membrane structure of Euglena is unique and different from those of other organisms. Although the mechanism of incorporation or uptake of radionuclide such as technetium in Euglena in the present invention is not clear but it may be considered that the membrane structure that is commonly observed in genus Euglena would contribute to the uptake of technetium.

Among Euglena species, those satisfying the following conditions are preferred; 1) species which prefer acidic condition; 2) species which can be easily cultured; 3) species which can rapidly proliferate; 4) species of which maximum population density is high. Further, Euglena gracilis is more preferable among the species that satisfy the above conditions.

Since uptake of radionuclide cannot be seen in dead cells of Euglena, it may be necessary to contact radionuclide with viable cells of Euglena.

Accordingly, in the present method, radionuclide is contacted with Euglena under the condition in which Euglena is viable. Examples of the condition in which Euglena is viable include those described in, for example, “Euglena physiology and biochemistry”, p. 1-3, edited by Kitaoka Seisaburo, Gakkai-Syuppan-Center, Dec. 10, 1989.

Since Euglena can be grown under aerobic condition, the method of the present invention can be conducted under aerobic condition. Thus, the present invention is advantageous over the conventional method of removing radionuclide by microorganisms that is conducted under anaerobic condition.

Examples of culture medium that is suitable for cultivation of Euglena include TGY medium, Cramer-Myers medium, Hutner medium, Koren-Hutner medium and the like. Preferably, TGY medium is used. Although most of these media for Euglena require a lot of organic materials, inorganic salts and minor elements in its synthesis, TGY medium has a simple composition made from triptone, yeast extract, glucose and vitamin B12. The cost and the time for preparing medium can be saved if TGY medium is used. In addition, cell density of Euglena gracilis reaches beyond the level of 10⁶ cells/ml in a short period of time in this medium.

Euglena usually can be grown at a wide range of pH from 3 to 8 by altering the condition of medium but it is preferable to grow Euglena at pH 7.0 or less, more preferably pH 6.5 or less, for use in the removal of technetium. In addition, incubation of Euglena at the pH condition that Euglena shows good growth, that is, around pH 3.5 (pH 3.0 to 4.0), is most preferable since radionuclide can be effectively removed. In this connection, pH of medium is generally adjusted suitably in the preparation of medium but it is known in the field of culture of organisms that the pH would be altered during the culture from the effects of contaminant or carbonate in the atmosphere. Therefore, it is understandable that the pH range described above would be altered at the same level as the change of pH during the usual culture.

The temperature condition is not limited to specific one but may be in the range wherein Euglena can be grown. Generally, the temperature can be raised up to approximately 34° C.

In the method of the present invention, “a solution in which Euglena is viable” means a solution in which Euglena can be grown and is under the conditions described above.

The amount of inoculation of Euglena is not limited to specific range but a higher cell density at the inoculation results in a higher removal rate of technetium. More specifically, the amount of inoculation is preferably higher than 10⁶ cell/ml.

Euglena can be grown autorophically, utilizing light as a source of energy while it also can be grown heterotrophically utilizing organic material as a source of energy. In this connection, a test for removing technetium using a bleached mutant of Euglena gracilis Z strain was conducted as described below. As the result, technetium could be removed by the mutant as well as Euglena gracilis.

Therefore, the light exposure may not be essential for the removal of technetium.

The bleached mutant was prepared as follows. Euglena was cultured at the condition of: 25° C., 2500 Lux, 12 hour-light-dark cycle, for one week in TGY medium (pH3.5) that was prepared by adding antibiotic Streptomycin so that the final concentration thereof becomes 500 μg/mL. After the incubation, the culture solution was applied on TYG agar plate (an agar plate medium that is prepared by adding 1.5% agar to TYG medium). After 10 days, white and yellow colonies on the agar were isolated. Normal Z strain forms a green colony on the agar medium. Each white and yellow colonies isolated were called “bleached mutant SmW” and “bleached mutant SmY”, respectively and were used in the experimental for the removal of technetium. The deficiency in the function of photosynthesis in “bleached mutant SmW” and “bleached mutant SmY” was confirmed by quantitative determination of chlorophyll a, which falls below the detection limit.

An example of radionuclide that can be removed by the method of the present invention includes technetium and the like.

By the method of the present invention, radionuclide, in particular technetium can be effectively removed. There are more than 20 kinds of isotopes for technetium, for example, ⁹⁷Tc, ⁹⁸Tc, ⁹⁹Tc and ⁹⁹mTc. All the isotopes emit radiation and thus are not stable. In the present specification, technetium includes all the isotopes.

Among the isotopes described above, ⁹⁹Tc is a fission product of Uranium and therefore it exists in large quantity in a nuclear spent fuel of atomic reactor. ⁹⁹Tc has an enormously long half-life as 210,000 years and is one of the most important nuclear species industrially. ⁹⁹Tc exists in nature mainly as pertechnetate ion (⁹⁹TcO₄⁻) that is easily soluble in water. In the present invention, technetium includes chemical species comprising technetium (⁹⁹Tc) such as pertechnetate (⁹⁹TcO₄⁻). Examples of other chemical species include [Tc(CO)]₅⁻, Tc²⁺, TcO₂⁻, TcO₂, TcO(OH)⁺, TcO(OH)₂, TcO₃⁻, TcF₆, TcO₃F, Tc₂O and the like.

In the method of the present invention, the separation of Euglena which contains a radionuclide from a solution can be conducted by the methods known as a separation method of solid phase and liquid phase such as filtration, decantation and the like. By the methods, radionuclide can be removed from a solution containing the radionuclide. When filtration is conducted, a filter having pore size which can collect Euglena may be used. For example, if Euglena gracilis is used, a filter having pore size of about 0.2 μm can be used for the collection of Euglena.

In the present invention, a composition for removing radionuclide comprising living cells of Euglena may be any composition comprising living cells of Euglena but more specifically, a composition comprising Euglena living cells in a solution such as culture solution. The above-described known components utilized for culture of Euglena can be used as a composition of culture solution.

### EXAMPLE

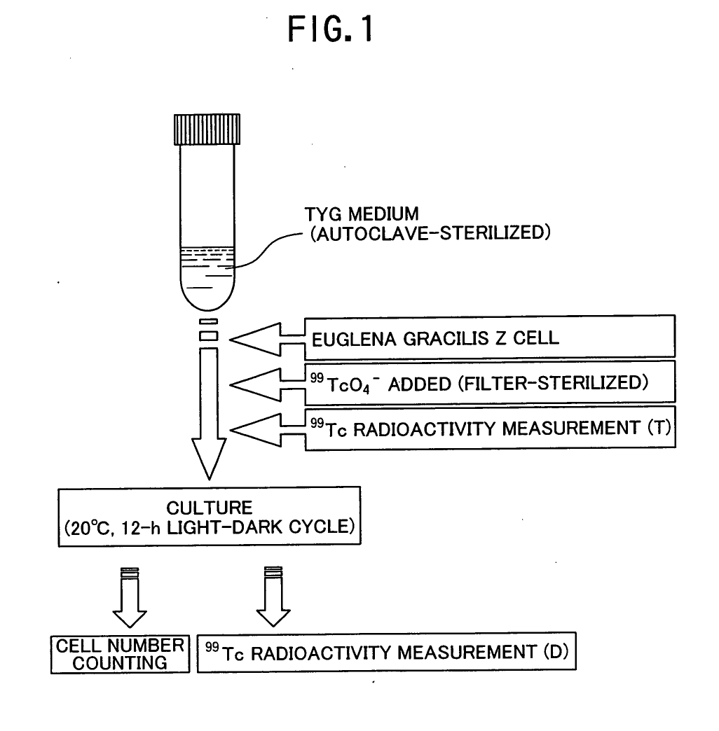

The following experiments were conducted. The summary of the procedure of the experiment was shown in FIG. 1.

1) Culture solution (TGY medium) comprising the components in Table 1 was prepared and was sterilized by autoclave (121° C., 15 minutes).

TABLE 1

| | | |
|---|---|---|
| Triptone | 1.0 | g |
| Yeast extract*¹ | 0.1 | g |
| Glucose | 1.0 | g |
| Vitamin B12 | 0.1 | μg |
| Distilled water | 100 | Ml |

| | |
|---|---|
| pH*² | 3.5 |

*¹bacto yeast extract manufactured by Difco Co., Ltd.
*²pH determined in the preparation of medium

2) A 30 mL of TYG medium was placed in a 50 mL tube for centrifuge separation (made of polypropylene).

3) Euglena gracilis Z strain was inoculated on the TYG medium. The amount of inoculation was about 10³-10⁶ cells/ml.

4) Filter sterilization of ⁹⁹Tc, which is in a form of ⁹⁹TcO₄⁻, i.e., ca. 0.01M NH₄TcO₄ in aqueous ammonia solution, was conducted through cellulose acetate filter having pore size of 0.2 μm and the solution was added in the culture solution inoculated with Euglena gracilis Z strain. The amount of ⁹⁹Tc was adjusted so that the radioactivity of ⁹⁹Tc in 100 μl of culture became 15000 cpm.

5) Stationary cultivation was conducted at 20° C. using 12 hour light-dark cycle.

6) The cell proliferation was determined by turbidity. The turbidity of algae was determined by measuring the absorption at 750 nm with HITACH U3210 spectrophotometer using a culture solution without the algae as a reference.

7) Radioactivity of ⁹⁹Tc was determined by the liquid scintillation counter as follows.

a) The culture solution was filtered through cellulose acetate filter having pore size of 0.2 μm.

b) A 100 μl of the filtrate was mixed with 4 ml of liquid scintillation counter cocktail.

c) The “cpm” was determined at 25 to 290 Kev.

Technetium that was added at the beginning of the culture is in the form of ⁹⁹TcO₄⁻, which was filter sterilized through a filter having pore size of 0.2 μm. Therefore, unless ⁹⁹TcO₄⁻ is changed physiochemically, the technetium should exist in the filtrate. However, the amount of ⁹⁹Tc in the filtrate was reduced as compared with the amount thereof at the beginning of the test. That is, Euglena gracilis Z strain cell adsorbed ⁹⁹TcO₄⁻ and thus ⁹⁹TcO₄⁻ was captured by filter with Euglena gracilis Z strain cell when the culture solution was filtered through the filter having pore size of 0.2 μm. In other words, ⁹⁹Tc was captured on the filter when the culture solution was filtered. In addition, since the half-life of ⁹⁹Tc added in the culture solution is as long as 210,000 years, the radioactivity per one element is not reduced during the culture period (for several months). Based on the above points, the relative amount of ⁹⁹Tc removed from a culture solution using Euglena gracilis Z strain can be determined in accordance with the following equation.

$$RRA = \frac{T - D}{T}$$

wherein, “RRA” means the relative amount of ⁹⁹Tc removed from the culture solution by Euglena gracilis Z strain, “T” means the radioactivity in the filtrate that is obtained by the filtration of culture solution immediately after the addition of ⁹⁹Tc, and “D” means ⁹⁹Tc radioactivity in the filtrate of culture solution after complete of cultivation.

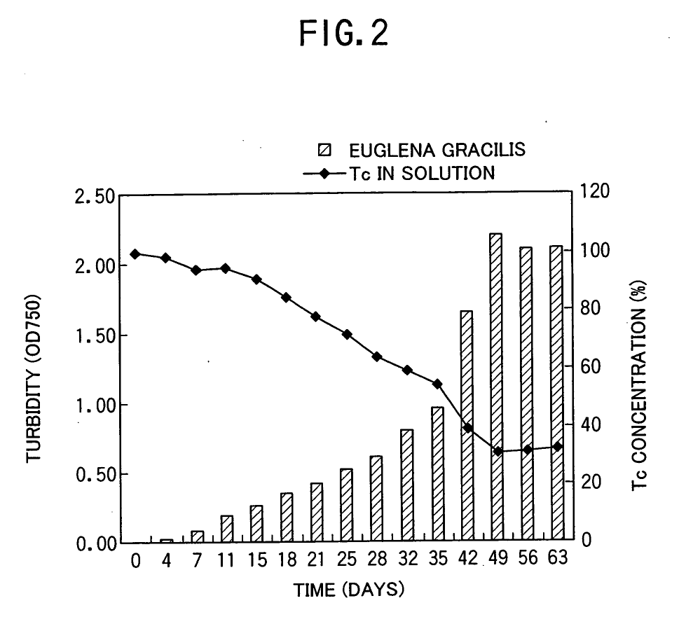

The changes of the number of Euglena cells and of the amount of ⁹⁹Tc in the culture solution with time were determined. As the results, it is found that Euglena cells during the growth phase to stationary phase can remove technetium from the solution (FIG. 2). In addition, it is found that technetium once incorporated in Euglena is maintained within the Euglena cell.

As comparative examples, the same experiments utilizing Chlorella vulgaris and Chlamydomonas pulsatilla were conducted but technetium could not be removed (data not shown).

### INDUSTRIAL APPLICABILITY

The present invention can be utilized for aerobic treatment of radioactive wastes.

What is claimed is:

1. A method for removing radionuclide from a solution characterized in use of Euglena.

2. A method for removing radionuclide from a solution comprising contacting radionuclide with Euglena in a solution in which Euglena is viable to thus transfer said radionuclide to Euglena and separating said Euglena containing the radionuclide from the solution.

3. The method of claim 1 or 2, wherein the radionuclide is technetium.

4. The method of claim 1 or 2, wherein Euglena is Euglenagracilis.

5. The method of claim 1 or 2, wherein the radionuclide is removed at pH of 3.0 to 6.5.

6. The method of claim 1 or 2, wherein the radionuclide is removed under aerobic condition.

7. A composition for removing radionuclide comprising a living cell of Euglena.
